(19) United States
(12) Patent Application Publication
(10) Pub. No.: US 2015/0057226 A1
(43) Pub. Date: Feb. 26, 2015

(54) METHOD FOR TREATMENT OF LABOR ARREST

(71) Applicant: DILAFOR AB, Solna (SE)

(72) Inventors: Gunvor Ekman-Ordeberg, Danderyd (SE); Anders Malmström, Lund (SE)

(21) Appl. No.: 14/387,929

(22) PCT Filed: Mar. 25, 2013
(86) PCT No.: PCT/SE2013/050333

§ 371 Date: Sep. 25, 2014

Related U.S. Application Data
(60) Provisional application No. 61/615,400, filed on Mar. 26, 2012.

(51) Int. Cl. A61K 31/727; A61K 31/737; A61K 38/22

(52) U.S. Cl. CPC A61K 31/727; A61K 38/22; A61K 31/737
USPC 514/11.6

(57) ABSTRACT

The present invention refers to the use of certain sulfated glycosaminoglycans for treatment of labor arrest. The sulfated glycosaminoglycans have a reduced anticoagulant activity and are administered in combination with an agent capable of promoting myometrial contractions of the uterus and thereby re-establish effective labor.

### FIELD OF INVENTION

The present invention refers to the use of certain sulfated glycosaminoglycans for treatment of labor arrest by re-establishing effective labor in women that during labor enters labor arrest.

### BACKGROUND

A common clinical problem in obstetrics is protracted or in some way dysfunctional labor. Slow progress or arrest of labor is documented in about 40-60% of all parturitions in for example Europe and in the USA. In developing countries labor arrest with heavy post partum bleeding are the most common reasons for maternal deaths.

The uterus is composed of two parts, the corpus and the cervix having different functions during pregnancy and parturition. The corpus uteri consist predominantly of smooth muscle bundles, the myometrium, embedded in extra cellular matrix, ECM, while the cervix consists mainly of ECM. The dominating components of the ECM are collagen I and III and proteoglycans albeit in a smaller quantities. Proteoglycans consist of a protein core to which one to a hundred polysaccharide chains, the glycosaminoglycans, are attached. The coordination between the uterine contractions and the softening, or in other words ripening, and dilation of the cervix is crucial for a normal parturition. Incongruity between these processes leads to abnormal parturitions.

In Acta Obstetricia et Gynecologica. 2010; 89: 147-150, it is reported that dalteparin, a Low Molecular Weight Heparin (LMWH) has been found to improve labor progress and thereby reduce the labor time and it is suggested that dalteparin increases the oxytocin induced uterine smooth muscle contractions and also stimulate the release of cytokines in cervix around partus. Even if dalteparin generally appears to cause positive effects on the labor process it would not be clinically feasible to use due to the risks for bleeding from its anticoagulant effect.

WO 03055499 teaches that sulfated glycosaminoglycans, such as heparin, having an anticoagulant activity of 100 BP units/mg or less, are effective for prophylactic priming or curative treatment of the cervix and the myometrium for establishing effective labor in women in general. In this document, it is suggested that sulfated glycosaminoglycans can be used in combination with oxytocin for the priming of the myometrium in cases of low endogenous oxytocin levels. It is however, not suggested that the sulfated glycosaminoglycans would be useful in directly intervening therapies when complications arise that require a direct therapeutic efficacy.

As of today, women experiencing labor arrest are administered oxytocin in increasing levels until effective labor is re-established. Due to the administration of a high dose of oxytocin labor in order to reestablish effective contractions, there is a high risk of too frequent contractions resulting in lower blood flow in the placenta jeopardizing the fetus and not seldom resulting in fetal asphyxia.

There have been few efforts to develop new drugs for labor augmentation despite a tremendous global problem of women entering labor arrest during labor. The present invention solves the problem by administering, to women in labor arrest, an effective amount of certain sulfated glycosaminoglycans in order to re-establish effective labor.

### SUMMARY OF THE INVENTION

The present invention relates to treatment of labor arrest. A chemically modified heparin or heparan sulfate with an antifactor IIa activity of less than 10 IU/mg and an antifactor Xa activity of less than 10 IU/mg is administered in combination with an agent capable of promoting myometrial contractions of the uterus and thereby re-establish effective labor and treat the labor arrest. Both primary and secondary arrest can be treated by the method and uses according to the present invention.

### BRIEF DESCRIPTION OF THE FIGURES

FIG. 1 is a graph showing the delivery time in women who received oxytocin and have been treated with a chemically modified heparin or heparan sulfate according to the invention (DF01) or received placebo

FIGS. 2A-2D show calcium ion influx in uterine muscle cells when treated with combinations of oxytocin and a chemically modified heparin or heparan sulfate according to the invention (DF01).

### DESCRIPTION OF THE INVENTION

Before the present invention is described, it is to be understood that the terminology employed herein is used for the purpose of describing particular embodiments only and is not intended to be limiting, since the scope of the present invention will be limited only by the appended claims and equivalents thereof.

It must be noted that, as used in this specification and the appended claims, the singular forms “a,” “an,” and “the” include plural referents unless the context clearly dictates otherwise.

Also, the term “about” is used to indicate a deviation of +/−2% of the given value, preferably +/−5%, and most preferably +/−10% of the numeric values, where applicable.

The term “labor arrest” is used in the context of the present invention to characterize abnormalities in labor during all stages of labor starting from once the pregnant woman is having repetitive uterine contractions. Normal progress of labor is defined as regular myometrial contractions of the uterus leading to a cervical dilatation of at least about 1 cm per hour until a dilatation of 10 cm. In the context of the present invention normal progress of labor is also defined as effective labor.

Labor arrest means conditions varying from a slower than normal progress (i.e. less than about 1 cm cervical dilatation during 1 hour, during 1-2 hours or during at least 2 hours) to a complete absence of progress of cervical ripening and myometrial contractions of the uterus. Labor arrest is more common among nulliparous than multiparous women. According to current practice, and after it has been established that the woman has a slower than normal progress, treatment with oxytocin is normally initiated after awaiting one additional hour in order to see if the woman can enter into normal progress without treatment.

A woman can enter into labor arrest at different stages of labor. Early stage labor arrest (sometimes called “primary arrest”) is often due to impaired cervical dilatation and in late phase of the labor (i.e. when the woman is dilated 5-6 cm and with a normal progress initially cm and called “secondary arrest”) due to impaired or insufficient myometrial contractions of the uterus. The meaning of labor arrest in this context extends to clinically common terms like dystocia, slow progress in labor, arrest of labor, complete cessation of progress, dysfunctional labor failure and cephalopelvic disproportion occurring after repetitive uterine contractions have been experienced. The onset of labor before arrest may be spontaneous or induced by conventional processes or therapies. There is a higher frequency of women experiencing labor arrest among women that have been induced into labor by pharmaceutical or physical means compared to women wherein labor onset was spontaneous.

In the context of the present invention the term “treatment of labor arrest” relates to a therapy where a direct response effect is requested from the administration. In the context of labor it is requested that the administration directly leads to promotion or stimulation of myometrial contractions of the uterus. In other terms, the present invention is not directed to a prophylactic therapy, wherein women may receive a therapy to prevent from or counteract protracted labor, before entering into labor.

The term “in combination” shall have meaning of a combined treatment with a chemically modified heparin or heparan sulfate described in the present invention and another agent that is effective in promoting or stimulating myometrial contractions of the uterus. “In combination” shall have a broad meaning and encompass any of the conditions that the treatments are performed adjunctively, simultaneously, sequentially or in parallel. It may also have the meaning of a chemically modified heparin or heparan sulfate described in the present invention and administered as an add-on treatment to another agent useful for promoting or stimulating myometrial contractions of the uterus. In case of an add-on treatment, a chemically modified heparin or heparan sulfate described in the present invention is administered to a woman already being treated with an agent capable of promoting myometrial contractions of the uterus.

Sulfated glycosaminoglycans with low anticoagulant effect, such as an anti-factor Xa activity below 200 IU/mg, are disclosed herein for re-establishing effective labor in women that are suffering from labor arrest. The sulfated glycosaminoglycans are administered as in combination with an agent capable of promoting myometrial contractions of the uterus in the treatment of labor arrest.

The glycosaminoglycans are sulfated glycosaminoglycans selected from the group consisting of heparan sulfate, depolymerized heparan sulfate, heparin, depolymerized heparin (e.g. low molecular weight heparin) dermatan sulfate, dermatan sulfate, chondroitin sulfates and depolymerized chondroitin sulfates.

The sulfated glycosaminoglycans are heparan sulfate, dermatan sulfate and chondroitin sulfate, which are composed of alternating hexosamine and uronic acid residue. The presence of D-glucuronic acid (GlcA) and its C-5 epimer L-iduronic acid (IdoA) and the specific sulfation of hexosamines and uronosyl residue endow the polymer an extreme structural variation. The structure is built on repeating disaccharides containing from none or very few to nearly 100% iduronic acid-containing disaccharides. The organization of GlcA- and IdoA-N-hexosamine containing disaccharides can vary from long blocks to an alternating disaccharide pattern. The variation of sulfation and the degree of iduronic acid sulfate generates a wide variety of biological activity. There are different well-defined polysaccharides of dermatan sulfate, chondroitin sulfate, heparan sulfate, heparin and heparin.

Heparan sulfate, having glucosamine and uronic acid as repeating disaccharides and consisting of N-acetylated and N-sulfated disaccharides that are arranged mainly in a segregated manner, has ubiquitous distribution on cell surfaces and in the extracellular matrix. It is generally less sulfated and has a lower iduronate content than heparin and has a more varied structure. Interactions between heparan sulfate and proteins are implicated in a variety of physiological processes, such as cell adhesion, cell proliferation, enzyme regulation, cytokine action, virus entry and anticoagulant properties. Heparan sulfates possess anticoagulant activity depending on the presence of a specific anticoagulant pentasaccharide, however considerably less than heparin. Heparan sulfate is a linear polysaccharide which can be prepared from porcine intestinal mucosa or from bovine lung, from heparin side fractions using cetylpyridinium chloride fractions and sequential salt extraction as described by Fransson et al., Structural studies on heparan sulphates, Eur. J. Biochem. 106, 59-69 (1980).

Chondroitin sulfate is a sulfate linear polysaccharide consisting of alternating glucuronic acid and N-acetyl-galactosamine residue, the latter being sulfate in either 4 or 6 position. They can be prepared from bovine trachea or nasal cartilage. Chondroitin sulfate is of importance for the organization of extracellular matrix, generating an interstitial swelling pressure and participating in recruitment of neutrophils.

Dermatan sulfate is a sulfate linear polysaccharide consisting of alternating uronic acid and N-acetyl-galactosamine residue. The uronic acids are either D-GlcA or L-IdoA and the disaccharide can be sulfate in 4 and 6 and 2 on galactosamine and IdoA, respectively. Dermatan sulfate can be prepared from porcine skin, intestinal mucosa and bovine lung. It possesses biological activities such as organization of extracellular matrix, interactions with cytokines, anticoagulant activities and recruitment of neutrophils.

Heparin is a naturally occurring glycosaminoglycan that is synthesized by and stored intracellulary in mast cells. Prepared industrially from porcine intestinal mucosa, heparin is a potent anticoagulant and has been used clinically for more than 60 years as the drug of preference for prophylaxis and treatment of thromboembolic disorders. The major potential adverse effects of heparin treatment are bleeding complications caused by its anticoagulant properties and low bioavailability. Heparin is highly polydisperse and composed of a heterogeneous population of polysaccharides with molecular weights ranging from 5 to 40 kDa, with the average being approximately 15 to 18 kDa.

Low molecular weight heparin or heparin is linear oligosaccharides mainly consisting of alternating N-sulfated glucosamine and IdoA residue and often containing an anticoagulant pentasaccharide. They can be prepared from heparin by specific chemical or enzymatic cleavage. Their main clinical function is to potentiate inhibition by antithrombin of coagulation factor Xa, resulting in an antithrombotic effect. It is proposed to have antimetastatic properties. Fragmin® (Pfizer, USA) is an example of a low molecular heparin obtained by controlled of heparin and having an antithrombotic effect owing to inhibition of factor Xa. Heparin fragments having selective anticoagulant activity, as well as methods for the preparation thereof, are described in U.S. Pat. No. 4,303,651. According to the European pharmacopoeia (PharmEur) a heparin in order to be called a low molecular weight heparin (low molecular mass heparin) should have an antifactor Xa activity not less than 70 IU (International Unit)/mg and an M_{w} of less than 8 000 Da. The anticoagulant activity of heparin, Low Molecular Weight Heparins and other heparin derivatives is often measured as their ability to potentiate the inhibition of coagulation factor Xa and factor IIa by antithrombin. Methods for measuring anti-factor Xa- and anti-factor IIa activity are well known to the skilled person and are also described in pharmacopoeias such as the European pharmacopoeia (Pharm Eur) and the United States pharmacopoeia (USP). The anticoagulant activity can be abrogated by for example selective periodate oxidation (see e.g. Fransson L A, and Lewis W, Relationship between anticoagulant activity of heparin and susceptibility, to periodate oxidation, FEBS Lett. 1979, 97: 119-23; Lindahl et al., Proc Natl Acad Sci USA, 1980; 77(11):6551-6555) but also by other means known to the skilled person.

Low molecular weight heparin or depolymerized heparin is a mixture of linear oligosaccharides mainly consisting of alternating N-sulfated glucosamine and IdoA residue and often containing the anticoagulant pentasaccharide. They can be prepared from heparin by specific chemical cleavage. Their main clinical function is to inhibit factor Xa, resulting in an antithrombotic effect. It is proposed to have antimetastatic properties. Fragmin® (Pfizer, USA) is an example of a low molecular heparin obtained by controlled of heparin and having an antithrombotic effect owing to inhibition of factor Xa. Heparin fragments having selective anticoagulant activity, as well as methods for the preparation thereof, are described in U.S. Pat. No. 4,303,651.

The present invention relates to a method for treatment of labor arrest in a woman entering into labor arrest after repetitive uterine contractions have been experienced. By administering to the pregnant woman in labor arrest an effective amount at least one chemically modified heparin or heparan sulfate with an antifactor IIa activity of less than 10 IU/mg and an antifactor Xa activity of less than 10 IU/mg in combination with an agent capable of promoting myometrial contractions of the uterus, effective labor is re-established and labor arrest thereby treated. In women experiencing primary labor arrest treatment of labor arrest means to regain repetitive myometrial contractions of the uterus of increasing frequency, duration, and strength causing normal progress of cervical dilatation and in women experiencing secondary labor arrest re-establishing effective labor and treatment of labor arrest means to regain cyclic myometrial contractions of the uterus leading to expulsion of the newborn child. The treatment of labor arrest according to the present invention shall be performed as a directly intervening administration therapy that directly after the administration initiates a process that directly or indirectly leads to the re-establishment of effective labor and treatment of labor arrest.

Labor arrest is associated with both low uterine concentrations and significantly decreased gene expression of heparan sulfate (Hjelm Cluff A, Byström B, Klimaviciute A, Dahlqvist C, Cebers G, Malmström A and Ekman-Ordeberg G: Prolonged labour associated with lower expression of syndecan 3 and connexin 43 in human uterine tissue. Reproductive Biology and Endocrinology 2006, 4:24). To the best knowledge of the present inventors administering a chemically modified heparin or heparan sulfate according to the present inventive method the concentration of heparan sulfate in the uterine smooth muscle can be restored and thus serves to treat labor arrest and thereby re-establish effective contractions. Treatment of labor arrest decreases the delivery time which in turn reduces the complications i.e. post partum haemorrhage and endometritis for the mother and increased risk of fetal asphyxia and infections associated with protracted labor time. Importantly, it also reduces the number of caesarian sections which are a surgical operation associated with risks both for the mother and the fetus. Caesarian sections are also costly and the present invention therefore also provides economical advantages.

Labor arrest can be associated with slow cervical ripening or with low frequency and ineffective contractions or both. The chemically modified heparin or heparan sulfate to be administered according to the invention can exert its effect both on the cervix and on the uterus. With regard to cervical ripening and to the best knowledge of the present inventors the chemically modified heparin or heparan sulfate to be administered according to the inventive method could also exert a synergistic effect with prostaglandinE2. With regard to uterine contractions and to the best knowledge of the present inventors the chemically modified heparin or heparan sulfate to be administered according to the invention will replenish the myometrial tissue levels with said chemically modified heparin or heparan sulfate so that, for example oxytocin (frequently used agent to induce labor) may exert its contractile effect on the myometrium. An effect will be that the amount of oxytocin administered can be reduced and thus its negative side effects can be avoided.

In one aspect, the chemically modified heparin or heparan sulfate administered according to the invention have a weight average molecular weight (Mw) of 30 000 Da or less. In another aspect the chemically modified heparin or heparan sulfate administered according to the invention have a weight average molecular weight (Mw) of less than 20 000 Da. In another aspect the chemically modified heparin or heparan sulfate administered according to the invention have a weight average molecular weight (Mw) of 10 000 Da or less. In another aspect the chemically modified heparin or heparan sulfate administered according to the invention have a weight average molecular weight (Mw) not higher than 8 000 Da. In yet another aspect the chemically modified heparin or heparan sulfate administered according to the invention have a weight average molecular weight (Mw) not higher than 7 000 Da.

In one aspect, the invention refers to a method wherein a chemically modified heparin belonging to the group consisting of heparin having an average molecular weight below 20 000 Da is administered to a woman suffering from labor arrest. In another aspect the depolymerized heparin have an average molecular weight below 10 000 Da. In another aspect the depolymerized heparin have an average molecular weight not higher than 8 000 Da and in yet another aspect the depolymerized heparin have an average molecular weight not higher than 7 000 Da.

As mentioned above some sulfated glycosaminoglycans have anticoagulant properties. For a preparation to be used, for example during labor to treat labor arrest by re-establish effective labor, an anticoagulant effect is, however, normally not desirable.

Thus, for applications where an anticoagulant effect is not desirable, the chemically modified heparin or heparan sulfate to be used in the method of the present invention has an anti-factor Xa activity of 30 IU/mg or less and an anti-factor IIa activity of 30 IU/mg or less. In another aspect the chemically modified heparin or heparan sulfate to be used in the method of the present invention has an anti-factor Xa activity of 10 IU/mg or less and an anti-factor IIa activity of 10 IU/mg or less.

The anticoagulant activity of heparin, Low Molecular Weight Heparins and other heparin derivatives is often measured as their ability to potentiate the inhibition of coagulation factor Xa and factor IIa by antithrombin. Methods for measuring anti-factor Xa- and anti-factor IIa activity are well known to the skilled person and are also described in pharmacopoeias such as the European pharmacopoeia (Pharm Eur) and the United States pharmacopoeia (USP).

The anticoagulant activity can be abrogated by for example selective periodate oxidation (see e.g. Fransson L A, and Lewis W, Relationship between anticoagulant activity of heparin and susceptibility, to periodate oxidation, FEBS Lett. 1979, 97: 119-23; Lindahl et al., Proc Natl Acad Sci USA, 1980; 77(11):6551-6555) but also by other means known to the skilled person.

In one aspect the chemically modified heparin or heparan sulfate to be used in the inventive method has an antifactor Xa activity of 10 IU/mg or less and an antifactor IIa activity of 10 IU/mg or less.

In another aspect the disaccharide structure of the chemically modified heparin or heparan sulfate is essentially devoid of non-sulfated glucuronic and iduronic units and having an anti-factor Xa activity of 10 IU/mg or less and an anti-factor IIa activity of 10 IU/mg or less.

In yet another aspect the chemically modified heparin is a low anticoagulant heparin with an anti-factor Xa activity of 10 IU/mg or less and an average molecular weight not higher than 8 000 Da or not higher than 7 000 Da.

In one aspect, the invention is directed to the uses of a chemically modified heparin; wherein the anticoagulant effect of heparin is eradicated by treatment with periodate to eliminate antithrombin binding affinities. One non-limiting way of obtaining such a chemically modified heparin is periodate oxidation followed by alkaline β-elimination of the product. This process leads elimination of the anticoagulant activity. The process disclosed in U.S. Pat. No. 4,990,502 (Lormeau et al) demonstrates one way of treating native heparin to selectively cleave the pentasaccharide sequences responsible for the anticoagulant effect and a following depolymerization that results in a low anticoagulant heparin with a an average molecular weight 5.8 to 7.0 kDa.

In one aspect, the chemically modified heparin to be used in the method according to the invention has an average molecular weight (Mw) from about 4.6 and 6.9 kDa.

In one aspect, the inventive method is directed to the use of a chemically modified heparin for treatment of labor arrest comprising

(i) polysaccharide chains essentially free of chemically intact saccharide sequences mediating the anticoagulant effect; and
(ii) polysaccharide chains corresponding to molecular weights between 1.2 and 12 kDa with a predominantly occurring disaccharide according to (Formula I),

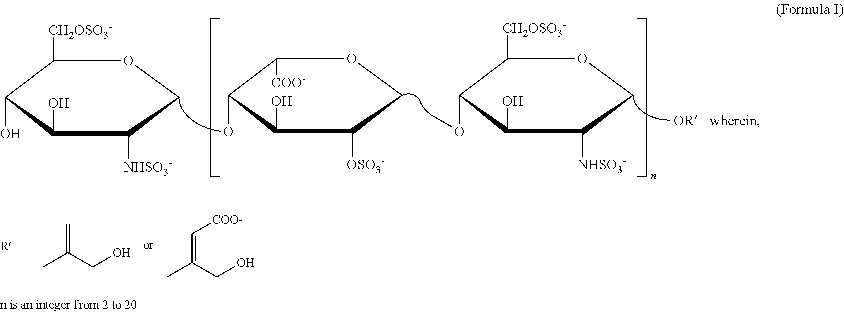

In this context, a chemically modified heparin or heparin sulfate, comprising polysaccharide chains essentially free of chemically intact saccharide sequences mediating the anticoagulant effect means that the polysaccharide chains have been treated chemically to modify essentially all the pentasaccharides specifically mediating an anticoagulant effect by antithrobmin (AT).

The predominantly occurring polysaccharide chains of such a chemically modified heparin have between 6 and 12 disaccharide units with molecular weights from 3.6-7.2 kDa, while at least 70% of the polysaccharide chains have a molecular weight above at least 3 kDa. The distribution of polysaccharides and their corresponding molecular mass expressed as cumulative % of weight would be according to the table:

| Molecular mass, kDa | Cumulative weight, % |
|---|---|
| >10 | 4-15 |
| >8 | 10-25 |
| >6 | 22-45 |
| >3 | >70 |

Furthermore, the polysaccharide comprises saccharide chains having the reduced end residue as shown in Formula I and is essentially free of intact non-sulfated iduronic and/or glucuronic acids.

In one aspect, this chemically modified heparin comprises modified glucosamines present as signals in the interval of 5.0 to 6.5 ppm in a ¹H-NMR spectrum with an intensity (% ratio) of less than 4% in relation to the signal at 5.42 ppm from native heparin. These glucosamine signals may be present at 5.95 ppm and 6.15 ppm. In one aspect, less than 1% of the total content of glucosamines is modified.

In this context, “modified glucosamines” has the meaning of glucosamines having a residue structure not expected to be found in a ¹H-NMR spectrum from heparin products or low molecular weight heparin products (depolymerized heparins). The appearance of modified glucosamines may be attributed to the chemical modification process for oxidizing non-sulfated iduronic and/or glucuronic acid in order to substantially eliminate the anticoagulant effect. It is desirable to minimize the presence of modified glucosamines as they may represent unpredictable characteristics of the chemically modified heparin product, such as non-specific depolymerization.

In one aspect, the chemically modified heparin comprises modified glucosamines in the non-reducing ends with unsaturated bonds. Such modified glucosamines are present as signals at 5.95 ppm and 6.15 ppm in an ¹H-NMR spectrum.

The method of the present invention can also be used to treat labor arrest regardless if the onset was induced or spontaneous. In the context of the present invention “labor induction” is generally defined as an intervention that directly or indirectly onsets a sufficiently effective labor from myometrical contractions of the uterus to accomplish a progress resulting in delivery and childbirth.

Labor can be induced in a number of ways, all well known to the skilled person. Non-limiting examples of methods to induce labor are physical stimulation processes; administration of oxytocin, prostaglandin E or derivatives thereof, such as misoprostol and dinoprostol; rupturing the amniotic sac; expanding the cervix, and administrating an intracervical ballon. Also combinations of these labor inducing processes can be used.

The present invention relates to a combined treatment with an agent capable of promoting or stimulating myometrial contractions of the uterus administered to the pregnant woman due to inadequate labor progress. Non-limiting examples of such an agent are oxytocin and prostaglandins like PGE1 (Misoprostol) and PGE2. In one aspect of the invention, the agent capable of promoting or stimulating myometrial contractions of the uterus is oxytocin. Thus, when the chemically modified heparin or heparan sulfate is administered as an adjuvant to oxytocin it promotes the oxytocin induced myometrial contractions of the uterus. The treatment regimen will be set by the skilled person and preferably set to fit with the clinical routines for oxytocin as the chemically modified heparin or heparan sulfate will be administered in parallel with oxytocin. In a non-limiting example of this aspect of the invention, the chemically modified heparin or heparan sulfate is administered at least once every 24 hours and adjunctively with a treatment with oxytocin for up to about 36 hours. In another aspect the chemically modified heparin or heparan sulfate is administered 1-24 times/24 h. In yet another aspect the chemically modified heparin or heparan sulfate is administered 6 times/24 h. The administration can be performed intravenously and/or subcutaneously. In one aspect the chemically modified heparin or heparan sulfate is administered by continuous infusion. Under current clinical practice oxytocin is administered intravenously.

In one aspect of the method, the women receive up to 1.5 g of the chemically modified heparin or heparan sulfate per 24 h. In another aspect, the women receive up to 1.2 g of the chemically modified heparin or heparan sulfate per 24 h and as a non-limiting example the 1.2 g/24 h is administered 6 times in doses of 200 mg. In one aspect of the method, the woman has experienced repetitive myometrial contractions but has entered into labor arrest. In this aspect, the method comprises administration of the chemically modified heparin or heparan sulfate that can be adjunct with an agent capable of promoting or stimulating uterine contractions, such as oxytocin, in order to regain the myometrial contractions.

In one aspect, the chemically modified heparin or heparan sulfate to be used with the invention can be formulated together with an effective amount of an agent promoting myometrial contractions of the uterus and thereby be administered together (co-administered) in one composition by previously suggested administration routes.

In one aspect, a composition of the chemically modified heparin or heparan sulfate to be used with the invention is included in a kit with at least a composition of an agent capable of promoting myometrial contractions of the uterus. The compositions can be provided in single- or multidose forms adapted to different clinical situations. The dose forms can be adapted to administration tools which also may be a part of the kit. For this purpose, the kit can further comprise clinical instructions how and when to administer the included compositions.

According to current practice the concentration of the agent promoting myometrial contractions is titrated in order to reach the desired effect and to not administer more than necessary of said agent to the woman. The titration usually starts with a low dose which is increased until the desired effect (i.e. myometrial contractions of the uterus) has been established. In one aspect, a composition of the chemically modified heparin or heparan sulfate is included in a kit together with a multidose form of at least a composition comprising an agent capable of promoting myometrial contractions of the uterus adapted to admit administration in several doses. In one example, the kit comprises a multidose form of oxytocin and the chemically modified heparin or heparan sulfate is administered in combination with an initial low or standardized dose of oxytocin. Should the patient remain in labor arrest, oxytocin may be administered one or several times with controlled doses from the multidose form until progress of labor is re-established.

The methods can comprise administration of the chemically modified heparin or heparan sulfates having the features as defined in any of the earlier parts of this specification.

Oxytocin is often administered to pregnant women to induce labor or to treat labor arrest. Frequently, the oxytocin effect is impaired, probably due to a lack of heparan sulfates leading to an over dosage of oxytocin that may result in severe side effects such as hyper contractility. The conjunctive use of the inventive method and administration of the chemically modified heparin or heparan sulfate can reverse the impaired oxytocin effect and thereby induce an oxytocin sparing effect and prevent the hypercontractility and the risk of fetal complications. To the best knowledge of the present inventors it could also be expressed in the following way: Oxytocin cannot exert its contractile effects unless necessary/adequate levels of heparan sulfates have been restored. Thus, the method and use according to the present invention leads to a reduced administration of oxytocin.

By treatment of labor arrest by re-establishing effective labor by the intervening therapy according to the invention, a shortened delivery time and the number of labor complications, e.g. caesarian sections can be significantly reduced. Protracted labor is also associated with other maternal complications e.g. post partum haemorrhage, instrumental deliveries and endometritis as well as an increased risk of fetal asphyxia and infection. Oxytocin's lack of effect on the uterine contractility results in frequent cesarean sections, including the ones performed on an emergency basis.

The chemically modified heparin or heparan sulfate to be used in the inventive method can be administered systemically as pharmaceutical compositions by parenteral administration, such as by subcutaneous or intravenous injection. For parenteral administration the active compounds can be incorporated into a solution or suspension, which also contain one or more adjuvants such as sterile diluents such as water for injection, saline, fixed oils, polyethylene glycol, glycerol, propylene glycol or other synthetic solvents, antibacterial agents, antioxidants, chelating agents, buffers and agents for adjusting the osmolarity. The parenteral preparation can be delivered in ampoules, vials, disposable syringes or as infusion arrangements, also for self administration.

The chemically modified heparin or heparan sulfate to be used in the inventive method can be administered subcutaneously and thereby also administered with suitable self-administration tools, such as injectors.

Further, the chemically modified heparin or heparan sulfate to be used in the inventive method are suitable for topical administration, including penetration of mucus membranes such as, but not limited to, vaginal, rectal, intra uterine, and nasal administration.

In one aspect the present invention also relates to a chemically modified heparin or heparan sulfate having an antifactor IIa activity of less than 10 IU/mg and an antifactor Xa activity of less than 10 IU/mg for use in combination with an agent capable of promoting myometrial contractions of the uterus in the treatment of labor arrest. All features disclosed above with regard to the inventive method and the chemically modified heparin or heparan sulfate also apply to this aspect of the invention.

In yet another aspect the present invention relates to the use of a chemically modified heparin or heparan sulfate having an antifactor IIa activity of less than 10 IU/mg and an anti-factor Xa activity of less than 10 IU/mg in combination with an agent capable of promoting myometrial contractions of the uterus for the manufacture of a medicament for use in the treatment of labor arrest. All features disclosed above with regard to the inventive method and the chemically modified heparin or heparan sulfate also apply to this aspect of the invention.

The present invention also relates to a method of reducing the administrated amount of oxytocin during labor, comprising the step of administering to a pregnant woman in labor suffering from labor arrest an effective amount of at least a chemically modified heparin or heparan sulfate having an antifactor IIa activity of less than 10 IU/mg and an anti-factor Xa activity of less than 10 IU/mg. All features disclosed above with regard to the inventive method and the chemically modified heparin or heparan sulfate also apply to this aspect of the invention.

Encompassed by the present invention is any combination of the embodiments and aspects disclosed in the present invention.

The invention will be further disclosed in the following non-limiting examples

### EXAMPLES

### Detailed Description of the Manufacturing Process of a Chemically Modified Heparin According to the Invention

The following examples 1 to 9 serve as examples how to produce chemically modified heparin or heparan sulfates for use according to the present invention.

The substance is prepared from sodium heparin. The preparation involves selective oxidation of non-sulfated uronic acid residues in heparin by periodate, including the glucuronic acid moiety in the pentasaccharide sequence that binds AT. Disruption of the structure of this residue annihilates the high-affinity interaction with AT and, consequently, the anticoagulant effect (measured as a-FXa or a-FIIa) is essentially depleted. Subsequent alkaline treatment, beta-elimination reaction results in cleavage of the polymer at the sites of non-sulfated uronic acids that have been oxidized by periodate. Together, these manipulations lead to a loss of anticoagulant activity along with adequate de-polymerization of the heparin chain.

Further, the resulting reducing end terminal at the site of cleavage is reduced by NaBH₄, which converts the terminal aldehyde to the corresponding diols which are more stable. Subsequently, additives, impurities and side-products are removed by repeated precipitations with ethanol, filtration and centrifugations. Thereafter the substance is obtained in powder form by drying with vacuum and heat. The drug substance will be dissolved in a sterile aqueous buffer to yield the drug product, which is intended for intravenous or subcutaneous administration.

The processes so far described generally include the steps of oxidation, polymer cleavage (alkaline hydrolysis) and reduction. The processes according to the present invention are developed in order to counteract or eliminate any type of non-specific depolymerization of the heparin chains. Non-specific polymerization in this context means generally such depolymerization that is not related to the specific alkaline beta-elimination reaction. Non-specific depolymerization results in structural instabilities of the product that may result in further depolymerization and discoloration during storage of the purified product. In addition, it may contribute to the appearance of atypical species appearing in NMR spectra not normally found in heparin.

The processes described and exemplified in the following section include different aspects of counteracting or eliminating non-specific depolymerization.

### Example 1

### Oxidation of Non-Sulfated Glucuronic- and Iduronic Acid (Residues), Deletion of AT-Binding Pentasaccharide and Anticoagulant Activity

A quantity of about 3000 grams of heparin is dissolved in purified water to obtain a 10-20% w/v solution. The pH of this solution is adjusted to 4.5-5.5. The sodium metaperiodate (NaIO₄) is subsequently added to the process solution; quantity of periodate 15-25% of the weight of heparin. The pH is again adjusted to 4.5-5.5. The reaction is protected from light. The process solution is reacted during the 18-24 hours with constant stirring maintenance of the temperature at 13-17° C., while the temperature is reduced to 5° C. during the last two hours.

### Termination of the Oxidation Reaction and Removal of Iodine-Containing Compounds

Ethanol (95-99.5%) is added to the reaction mixture over a period of 0.5-1 hour, with careful stirring and at a temperature of 5-25° C. The volume of ethanol to be added is in the range 1-2 volumes of ethanol per volume of process solution. The oxidized heparin is then allowed to precipitate and sediment for 15-20 hours, after which the mother liquor is decanted and discarded.

Next, the sediment is dissolved in purified water to obtain a 15-30% w/v process solution. NaCl is added to obtain a concentration of 0.15-0.30 mol/liter in the process solution. Stirring continues for another 0.5-1 hour while maintaining the temperature of 5-25° C. Subsequently 1.0-2.0 volumes of ethanol (95-99.5%) per volume of process solution are added to this solution with stirring, during a period of 0.5-1 hour. This precipitates the product from the solution.

### De-Polymerization of Polysaccharide Chains by an Alkaline Beta Elimination Process

After the mother liquor has been decanted and discarded, the sediment is stirred in approximately 7 litres of water until completely dissolved, the concentration of the solution is now 15-30%. While maintaining the temperature at 5-25° C. a 4 M NaOH solution is added slowly until a pH of 10.5-12 is obtained. The reaction is initiated and proceeds for 15-95 minutes. At this time, the pH of the solution is recorded and 4 M HCl is added slowly until a pH of 5.5-7 is obtained.

### Reduction of Reducing End Terminals

While maintaining the temperature at 13-17° C., the pH of the solution is adjusted to 5.5-6.5. A quantity of 130-150 grams of sodium borohydride is then added to the solution while the pH will increase to 10-11, the reaction is continued for 14-20 hours. After this reaction time, a dilute acid is added slowly in order to adjust the pH to a value of 4, this degrades remaining sodium borohydride. After maintaining a pH of 4 for 45-60 minutes, the pH of the solution is adjusted to 7 with a dilute NaOH solution.

The purification continues according to example 5

### Example 2

### Oxidation of Glucuronic and Iduronic Acid (Residues), Deletion of Anticoagulant Activity

A quantity of about 3000 grams of heparin is dissolved in purified water to obtain a 10-20% w/v solution. The pH of this solution is adjusted to 4.5-5.5. The sodium metaperiodate (NaIO₄) is subsequently added to the process solution; quantity of periodate 15-25% of the weight of heparin. The pH is again adjusted to 4.5-5.5. The reaction is protected from light. The process solution is reacted during the 22-26 hours with constant stirring and maintenance of the temperature at 13-17° C., while the temperature is reduced to 5° C. during the last two hours. The pH at the end of the reaction period is measured and recorded.

### Termination of the Oxidation Reaction and Removal of Iodine-Containing Compounds

Ethanol (95-99.5%) is added to the reaction mixture over a period of 0.5-1 hour, with careful stirring and at a temperature of 5-25° C. The volume of ethanol to be added is in the range 1-2 volumes of ethanol per volume of process solution. The oxidized heparin is then allowed to precipitate and sediment for 15-20 hours, after which the mother liquor is decanted and discarded.

### De-Polymerization of Polysaccharide Chains by an Alkaline Beta Elimination Process

After the mother liquor has been decanted and discarded, the sediment is stirred in approximately 7 litres of water until it appears visually to be completely dissolved. While maintaining the temperature at 20-25° C. 4 M NaOH is added slowly until a pH of 10.5-12 is obtained and the reaction thus initiated is allowed to proceed for 15-95 minutes. At this time, the pH of the solution is recorded and 4 M HCl is added slowly until a pH of 5.5-7 is obtained.

### Reduction of Reducing End Terminals

After the mother liquor has been decanted and discarded, the sediment is dissolved by addition of purified water until a concentration of the process solution of 15-30% w/v is obtained. While maintaining the temperature at 13-17° C., the pH of the solution is adjusted to 5.5-6.5. A quantity of 130-150 grams of sodium borohydride is then added to the solution and dissolved, the pH will immediately increase to a pH of 10-11, the reaction is continued for 14-20 hours. The pH of the solution, both prior to and after this reaction period, is recorded. After this reaction time, a dilute acid is added slowly in order to adjust the pH to a value of 4, this degrades remaining sodium borohydride. After maintaining a pH of 4 for 45-60 minutes, the pH of the solution is adjusted to 7 with a dilute NaOH solution.

Purification continues according to Example 5.

### Example 3

### Oxidation of Glucuronic and Iduronic Acid (Residues), Deletion of Anticoagulant Activity

A quantity of about 3000 grams of heparin is dissolved in purified water to obtain a 10-20% w/v solution. The pH of this solution is adjusted to 4.5-5.5. The sodium metaperiodate (NaIO₄) is subsequently added to the process solution, quantity of periodate 15-25% of the weight of heparin. The pH is again adjusted to 4.5-5.5. The reactor is protected from light. The process solution is reacted during the 18-24 hours with constant stirring maintenance of the temperature at 13-17° C., while the temperature is reduced to 5° C. during the last two hours.

### De-Polymerization of Polysaccharide Chains by an Alkaline Beta Elimination Process

While maintaining the temperature at 5-25° C., 4 M NaOH solution is added slowly until a pH of 10.5-12 is obtained. The reaction is initiated and proceeds for 15-95 minutes. At this time, the pH of the solution is recorded and 4 M HCl is added slowly until a pH of 5.5-7 is obtained.

### Reduction of Reducing End Terminals

While maintaining the temperature at 13-17° C., the pH of the solution is adjusted to 5.5-6.5. A quantity of 130-200 grams of sodium borohydride is then added to the solution while the pH will increase to 10-11, the reaction is continued for 14-20 hours. After this reaction time, a dilute acid is added slowly in order to adjust the pH to a value of 4, this degrades remaining sodium borohydride. After maintaining a pH of 4 for 45-60 minutes, the pH of the solution is adjusted to 7 with a dilute NaOH solution.

### Precipitation of Reduced Product and Initial Removal of Iodine-Containing Compounds

Ethanol (95-99.5%) is added to the reaction mixture over a period of 0.5-1 hour, with careful stirring and at a temperature of 5-25° C. The volume of ethanol to be added is in the range 1-2 volumes of ethanol per volume of process solution. The oxidized heparin is then allowed to precipitate and sediment for 15-20 hours, after which the mother liquor is decanted and discarded.

Next, the sediment is dissolved in purified water to obtain a 15-30% w/v process solution. NaCl is added to obtain a concentration of 0.15-0.30 mol/liter in the process solution

Purification continues according to Example 5.

### Example 4

### Oxidation of Glucuronic and Iduronic Acid (Residues), Deletion of Anticoagulant Activity

A quantity of about 3000 grams of heparin is dissolved in purified water to obtain a 10-20% w/v solution. The pH of this solution is adjusted to 4.5-5.5. The sodium metaperiodate (NaIO₄) is subsequently added to the process solution, quantity of periodate 15-25% of the weight of heparin. The pH is again adjusted to 4.5-5.5. The reactor is protected from light. The process solution is reacted during the 18-24 hours with constant stirring maintenance of the temperature at 13-17° C., while the temperature is reduced to 5° C. during the last two hours. Next, glycerol is added to quench the reaction, i.e. to convert residual periodate to iodate, 150-200 ml of a 85% glycerol solution is added and reacted for 30-60 minutes while stirring.

### Precipitation of Product Removal of Iodine-Containing Compounds and Quencher/Reaction Products

Ethanol (95-99.5%) is added to the reaction mixture over a period of 0.5-1 hour, with careful stirring and at a temperature of 5-25° C. The volume of ethanol to be added is in the range 1-2 volumes of ethanol per volume of process solution. The oxidized heparin is then allowed to precipitate and sediment for 15-20 hours, after which the mother liquor is decanted and discarded.

Next, the sediment is dissolved in purified water to obtain a 15-30% w/v process solution. NaCl is added to obtain a concentration of 0.15-0.30 mol/liter in the process solution. Stirring continues for another 0.5-1 hour while maintaining the temperature of 5-25° C. Subsequently 1.0-2.0 volumes of ethanol (95-99.5%) per volume of process solution are added to this solution with stirring, during a period of 0.5-1 hour. This precipitates the product from the solution.

### De-Polymerization of Polysaccharide Chains by an Alkaline Beta Elimination Process

After the mother liquor has been decanted and discarded, the sediment is stirred in approximately 7 litres of water until it appears visually to be completely dissolved. While maintaining the temperature at 5-25° C. 4 M NaOH is added slowly until a pH of 10.5-12 is obtained and the reaction thus initiated is allowed to proceed for 60-95 minutes. At this time, the pH of the solution is recorded and 4 M HCl is added slowly until a pH of 5.5-7 is obtained.

### Reduction of Reducing End Terminals

After the mother liquor has been decanted and discarded, the sediment is dissolved by addition of purified water until a concentration of the process solution of 15-30% w/v is obtained. While maintaining the temperature at 13-17° C., the pH of the solution is adjusted to 5.5-6.5. A quantity of 130-150 grams of sodium borohydride is then added to the solution and dissolved, the pH will immediately increase to a pH of 10-11, the reaction is continued for 14-20 hours. The pH of the solution, both prior to and after this reaction period, is recorded. After this reaction time, a dilute acid is added slowly in order to adjust the pH to a value of 4, this degrades remaining sodium borohydride. After maintaining a pH of 4 for 45-60 minutes, the pH of the solution is adjusted to 7 with a dilute NaOH solution.

Purification proceeds according to Example 5.

### Example 5

### Purification of the Product

### Removal of Process Additives and Impurities, Addition of Counter-Ions and Filtration

Process solutions according to Examples 1-4 arriving from the final chemical modification step of reducing the end terminals by borohydride is worked up according the methodologies outlined below.

One volume of process solution is then added to 1.5-2.5 volumes of ethanol (95-99.5%) followed by centrifugation at >2000 G, at <20° C. for 20-30 minutes, after which the supernatant is decanted and discarded.

The product paste obtained by centrifugation is then dissolved in purified water to obtain a product concentration 10-20% w/v. Then NaCl is added to obtain a concentration of 0.20-0.35 mol/liter. Next 1.5-2.5 volumes of ethanol (95-99.5%) are added per volume of process solution which precipitates the product from the solution. Centrifugation follows as described above.

Next the remaining paste is added purified water to dissolve. The product concentration would now be in the range of 10-20% w/v. The pH of the product solution is now adjusted to 6.5-7.5. The solution is then filtered to remove any particulates. Then, to one volume of process solution is added 1.5-2.5 volumes of ethanol (95-99.5%). Centrifugation follows at >2000 G, and at <20° C. for 20-30 minutes after which the supernatant is decanted and discarded.

### Dewatering of Precipitate Paste and Reduction of Particle Size.

A reactor is filled with ethanol, volume about 2 liters. While stirring the ethanol, the precipitate paste is added. The mechanical stirring solidifies the paste and replaces the water present by the ethanol giving a homogenous particle suspension. The stirring is discontinued after 1-2 hours after which the particles are allowed to sediment. After removal of excessive liquid, the particles are passed through a sieve or a mill to obtain smaller and uniform sized particles.

### Drying of Product

The product is distributed evenly onto trays, and placed in a vacuum cabinet. Vacuum is applied and heating is performed at 35-40° C. A stream of nitrogen is passed through the drier at this time while maintaining the low pressure in the dryer. When a constant weight is obtained of the product, i.e. no further evaporation is noticed, the drying is considered complete. The product is packed and protected from humidity.

### Example 6

### Oxidation of Glucuronic and Iduronic Acid (Residues), Deletion of Anticoagulant Activity

A quantity of about 3000 grams of heparin is dissolved in purified water to obtain a 10-20% w/v solution. The pH of this solution is adjusted to 4.5-5.5. The sodium metaperiodate (NaIO₄) is subsequently added to the process solution, quantity of periodate 15-25% of the weight of heparin. The pH is again adjusted to 4.5-5.5. The reaction is protected from light. The process solution is reacted during the 18-24 hours with constant stirring maintenance of the temperature at 13-17° C., while the temperature is reduced to 5° C. during the last two hours.

### De-Polymerization of Polysaccharide Chains by an Alkaline Beta Elimination Process

While maintaining the temperature at 5-25° C. 4 M NaOH is added slowly until a pH of 10.5-12 is obtained and the reaction thus initiated is allowed to proceed for 15-95 minutes. At this time, the pH of the solution is recorded and 4 M HCl is added slowly until a pH of 5.5-7 is obtained.

### Reduction of Reducing End Terminals

After the mother liquor has been decanted and discarded, the sediment is dissolved by addition of purified water until a concentration of the process solution of 15-30% w/v is obtained. While maintaining the temperature at 13-17° C., the pH of the solution is adjusted to 5.5-6.5. A quantity of 130-200 grams of sodium borohydride is then added to the solution and dissolved, the pH will immediately increase to a pH of 10-11, the reaction is continued for 14-20 hours. The pH of the solution, both prior to and after this reaction period, is recorded. After this reaction time, a dilute acid is added slowly in order to adjust the pH to a value of 4, this degrades remaining sodium borohydride. After maintaining a pH of 4 for 45-60 minutes, the pH of the solution is adjusted to 7 with a dilute NaOH solution. Purified water is now added to the solution until a conductivity of 15-20 mS/cm is obtained of the reaction solution.

### Purification of Product by Anion Exchange Chromatography

A column with a diameter 500 mm is packed with media, DEAE-Sepharose or QAE-Sepharose to a volume of 25-30 liters corresponding to a bed height of 10-15 cm. The chromatography is performed in 3-4 cycles to consume the entire product.

Next buffers are prepared,

Equilibration buffer, Buffer A, 15 mM phosphate, 150 mM NaCl

Elution buffer, Buffer B, 2 M NaCl solution

Sanitation buffer, 0.5 M NaOH

The chromatography step is performed at 15-25° C., at flow rate of <200 cm/hour or approx. 350 liters/hour.

The column is equilibrated with the equilibration buffer until the eluent has a conductivity of 15-20 mS/cm. Next the oxidized heparin solution is pumped into the column. The quantity of crude product to be applied corresponds to <40 g/liter of chromatography media.

An isocratic wash follows with equilibration buffer and is discontinued when the UV 210-254 nm has reached a baseline. Typically 5 bed volumes of buffer are required to reach baseline. Chemicals added to the process and products formed of these are removed.

Next, the ionic strength of the buffer applied onto the column is linearly increased by performing a gradient elution. The Buffer A decreases from 100% to 0% replaced by 100% Buffer B over 5 bed volumes. The product, eluate is collected when the UV absorbance is >0.1 AU and is discontinued when the signal is <0.1 AU. Sanitation of the column is then performed after which it is again prepared for the next cycle of chromatography. Eluates from all runs are combined and stored at 15-25° C.

### De-Salting of the Product

One volume of the combined eluates from previous step is added 3 volumes of 95-99.5% ethanol, 15-25° C., under constant stirring. This precipitates the product out of solution. The product is allowed to sediment for >3 hours. Next, the sediment is dissolved in purified water to a concentration of 15-25%. The solution is now added to cold ethanol (<−5° C.) 95-99.5%, typically 5 volumes of ethanol per one volume of product solution are consumed. Next follows centrifugation in a continuous mode, >2000 G, the product paste is thereafter collected and prepared for drying.

### Drying of Product

The product is distributed evenly onto trays, and placed in a vacuum cabinet. Vacuum is applied and heating is performed at 35-40° C. A stream of nitrogen is passed through the drier at this time while maintaining the low pressure in the dryer. When a constant weight is obtained of the product, i.e. no further evaporation is noticed, the drying is considered complete. The product is milled and made homogenous, thereafter packed and protected from humidity.

### Example 8

Low anticoagulant heparin produced according to the examples 1 and 3 was subjected to 1H-NMR analysis and compared to the spectrum of native heparin.

Table II demonstrates signals in the interval 5.00 ppm to 6.50 ppm not present in native heparin generated from non-reducing end unsaturated glucosamines. The results of Table II show that it is possible to reduce the presence of such compounds not predicted to be present in spectrum from native heparin to low levels. In comparison, the current limit applicable to heparin quality control, monograph 7, EDQM is <4% compared to the signal at 5.42 ppm for any signal in the region 5.70-8.00 ppm.

TABLE II
Qualitative results of a low anticoagulant heparin
with regards to unusual signals. Signal intensity
for signals 6.15 and 5.95 ppm in a 1H-NMR spectra

| | | Intensity (% ratio) to 5.42 ppm signal of a native | |
|---|---|---|---|
| | | heparin following EDQM, monograph 7 | |
| | Production | 6.15 ppm % | 5.95 ppm % |
| Sample | method | of ref. signal | of ref. signal |
| Batch 1 | Example 1 | 11 | 12 |
| Batch 2 | Example 1 | 13 | 16 |
| Batch 3 | Example 3 | 2 | 2 |

Further, the presence of non reducing end unsaturated glucosamines was also quantified by combined 1H-NMR and 13C-NMR spectra evaluation (HSQC) and demonstrated as mol % of total glucosamines (see Table III).

Furthermore, the sample was analyzed by following the NMR two-dimensional (2D) method involving the combined use of proton and carbon NMR spectroscopy (HSQC) as previously described (see Guerrini M., Naggi A., Guglieri S, Santarsiero R, Torri G. Anal Biochem 2005; 337, 35-47.)

Table III demonstrates the fraction (%) of modified glucosamines compared to the total amount of glucosamines of the low anticoagulant heparin as present as signals at 5.95 ppm and 6.15 ppm in the ¹H-NMR spectrum.

TABLE III
Results from quantitative determination of unusual
signals 5.95 ppm, 6.15 ppm of total glucosamine

| | | 6.15 ppm signal | 5.95 ppm signal |
|---|---|---|---|
| | Production | mol % of | mol % of |
| Sample | method | glucosamine | glucosamine |
| Batch 1 | Example 1 | 6 | 3 |
| Batch 2 | Example 3 | <1 | <1 |

### Example 9

The product manufactured according to any one of the examples above can prepared as drug product by a conventional aseptic process, such as solution comprising 150 mg/mL of active product and Na phosphate to 15 mM, pH 6-8. The so obtained drug product is intended primarily for subcutaneous administration but suitable for intra-venous administration.

The resulting product is a depolymerized form of heparin with a projected average molecular weight of 4.6-6.9 kDa and with essentially no anticoagulant activity.

The product has a size distribution of polysaccharide polymers, with a range for n of 2-20 corresponding to molecular weights of 1.2-15 kDa. The predominant size is 6-16 disaccharide units corresponding to molecular weights of 3.6-9.6 kDa.

The molecular weight was determined by GPC-HPLC carried out with a TSK 2000 and TSK 3000 SW columns in series. Refractive index was used for evaluation. First international calibrant for LMWH was used.

Below is presented the molecular mass distribution and the corresponding part of the cumulative percentage of total weight.

TABLE IV
Distribution of polysaccharides and their corresponding molecular
mass in as cumulative % of weight for several batches

| Molecular mass, kDa | Cumulative weight, % |
|---|---|
| >15 | <1 |
| >10 | 4-15 |
| >9 | 7-20 |
| >8 | 10-27 |
| >7 | 15-35 |
| >6 | 22-45 |
| >5 | 34-56 |
| >4 | 47-70 |
| >3 | >70 |
| >2 | >85 |

The corresponding value for weight average molecular weight, Mw falls in the range 4.6-6.9 kDa

### Example 10

The stability of the drug substance (powder) and drug product dissolved in aqueous phosphate buffered solution of a chemically modified heparin produced in accordance with Examples 1 to 3 and formulated in accordance with Example 9 was studied for stability over 36 months at ambient temperature. The initial product was clear white to slight yellow solution had an absorbance at 400 nm (10% w/v solution) of 0.14, a pH of 7.0 and osmolality of 658 mOsm/kg, an average molecular weight of 5.6 kDa and a content of 150 mg/ml.

After 36 months, the drug product had the same visual appearance, an absorbance at 400 nm (10% w/v solution) of 0.13, a pH of 7.1 and osmolality of 657 mOsm/kg, an average molecular weight of 5.4 kDa and a content of 153 mg/ml.

### Example 11

### Subcutaneous Administration

Chemically modified heparin produced by the method disclosed in example 1 was labeled with tritium and administered to Sprauge Dawley rats and dogs.

### Results:

Following subcutaneous administration at 2, 8 and 24 mg heparin/kg/day in the rat and 3, 15 and 45 mg heparin/kg/day in the dog, absorption was rapid and maximal plasma levels were generally reached within 0.5 and 1.5 h in the rat and dog, respectively. The subcutaneous bioavailability was around 90% in both the rat and the dog. Interestingly, the corresponding bioavailability for heparin is about 10%.

### Example 12

### Treatment with DF01 During Late Pregnancy

### Study Design

This was a randomized, double blind, placebo-controlled, multicentre study to assess the safety and efficacy of pre-treatment with DF01 during late pregnancy in reducing labor time. Eighteen study centers in Sweden participated in the study.

DF01 is a chemically modified heparin according to the invention that is low-anticoagulant heparin chemically generated by periodate oxidation of heparin from pig intestinal mucosa, followed by β-elimination of the product following Examples 1 and 9.

The protocol stated that each subject would come to the clinic daily from the treatment start at a gestational age from week 38+0 up to week 40+0 until labor to receive a s.c. injection of the investigational medicinal product. The anticipated duration of participation in the study was 1-28 days (+screening and follow-up periods) for each subject. All women had to be induced into at the latest at 42+0 weeks of gestation. A maximum of 28 days of treatment [maximally 28 doses of the investigational medicinal product (IMP)] was given. A follow-up visit was to take place at 8-16 weeks after delivery.

### Treatments

DF01 is a depolymerized heparin that is essentially deprived of its anticoagulant activity (<10 IU/mg by pharmacopoeial anti-factor Xa- and anti-factor IIa assays). The weight average Mw is 5 000-7 000.

DF01 and matching placebo, were provided as solutions for subcutaneous injection.

The pharmaceutical preparation of DF01 is a solution for subcutaneous injection, 8 mL dispensed in glass vials sealed with a rubber stopper and covered with a tear-off aluminum cap.

Each mL of the DF01 solution contains the following:
DF01, 150 mg
Phosphate buffer, 0.015 M
Benzyl alcohol, 14 mg.

A sterile physiological sodium chloride solution preserved with benzyl alcohol was used as placebo. Eight (8) mL of the placebo were provided in vials in the same way as for the drug product.

Each mL of the placebo solution contains the following:
Sodium chloride, 9 mg
Benzyl alcohol, 14 mg.

The subjects received 60 mg/day of DF01 (0.4 mL) (corresponding to 1.00 mg/kg/day in a 60 kg subject) or placebo (0.4 mL).

The products was administered by daily subcutaneous injections with treatment start at gestational age of week 38+0 to week 40+0 and treatment duration until labor. If still undelivered at 42+0 labor was to be induced. The maximum duration of treatment was 28 days. The allowed time interval between the daily injections was 24+/−6 hours, i.e. 18-30 hours. If the time limits were occasionally not met or a dose missed, the treatment could still continue.

### Results

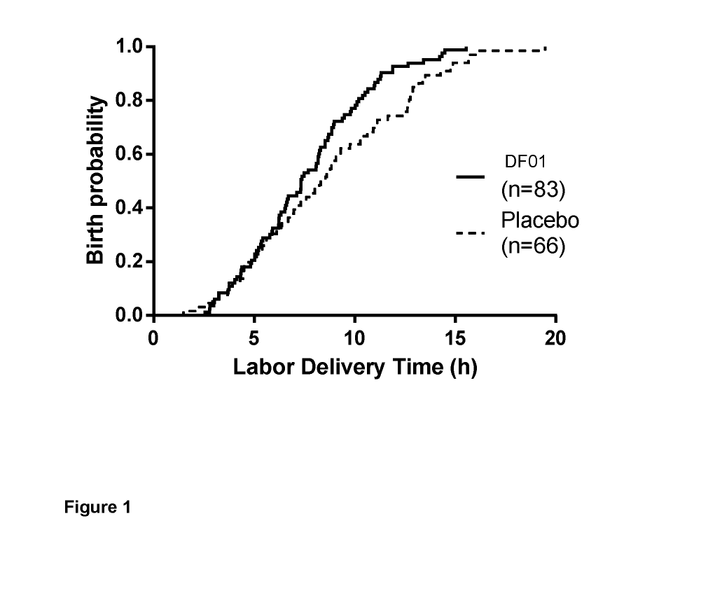

There were a total of 149 non-caesarean deliveries where the women received oxytocin (83 in the DF01 group an 66 in the Placebo group). The log-rank test showed a significant difference in delivery time between the treatment groups with a p-value of 0.0158. The product-limit birth curve is given in FIG. 1 and should be interpreted as follows. When labor lasts up to approximately 6-8 hours, the women will not receive oxytocin in a high number of cases and DF01 on its own seem to have minimal effect (the two curves follow each other closely). However, when the labor prolongs for more than 6-8 hours and the women are commonly given oxytocin, the additional administration of DF01 seems to potentiate the effect of oxytocin and facilitate a shorter labor time.

The inability of oxytocin to exert its effect is probably due to the lack of heparan sulfates and in many cases that leads to an overdosage of oxytocin that may result in severe side effects such as hyper contractility. The conjunctive use of DF01 can induce an oxytocin sparing effect and prevent the hypercontractility and the risk of fetal complications.

### Example 11

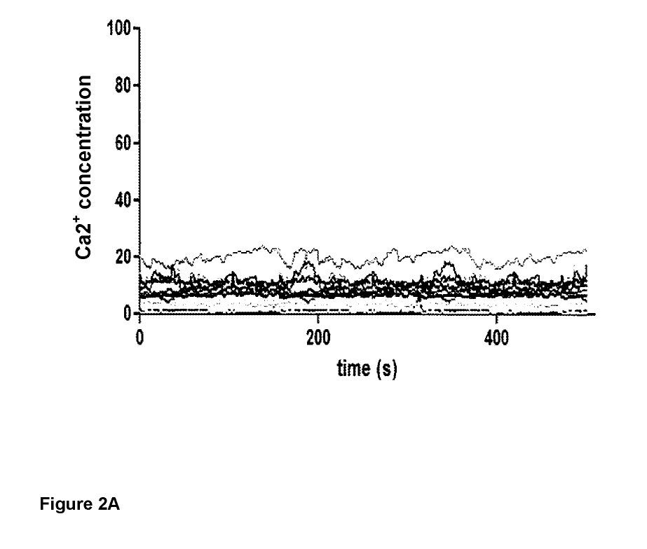

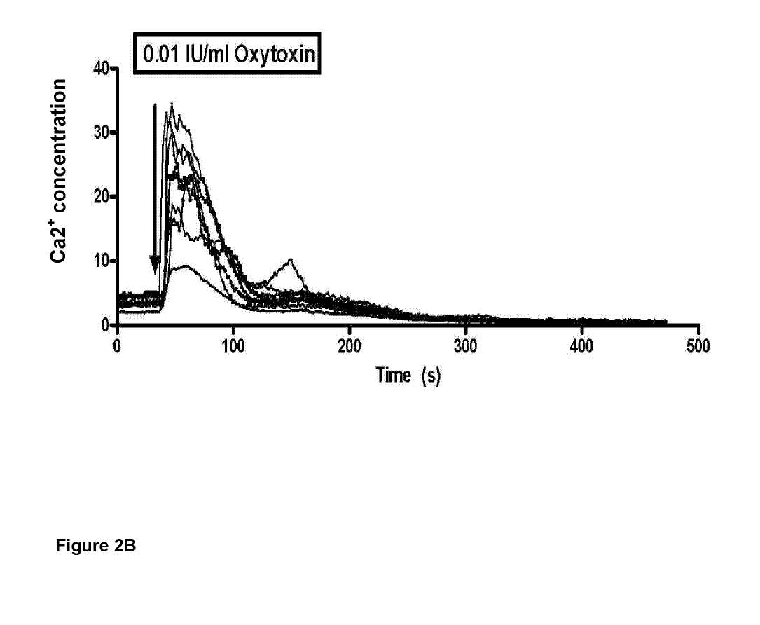

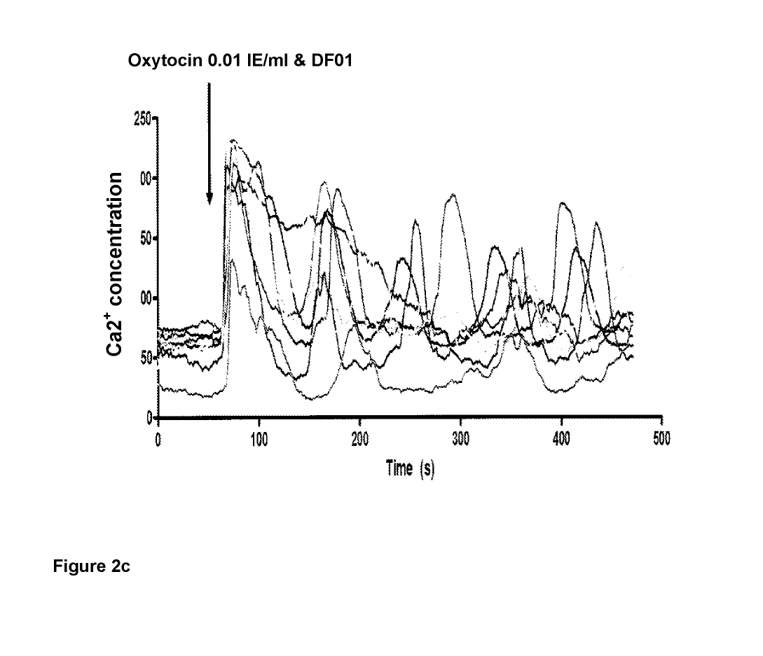

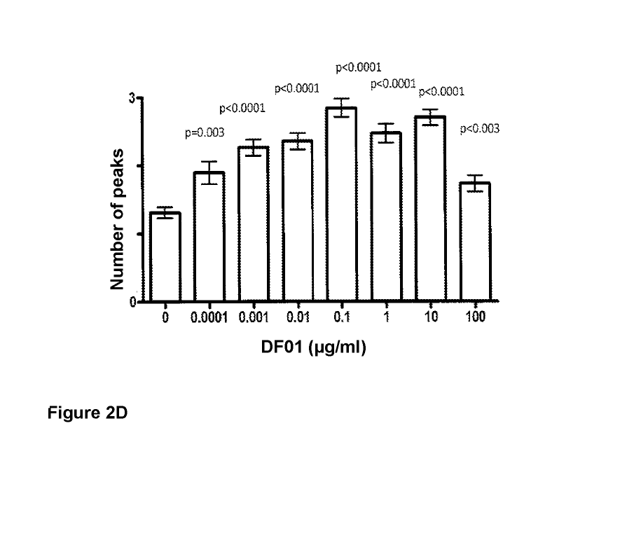

Human uterine smooth muscle cells were established in a culture. Intracellular Ca²⁺ was measured with the calcium indicator dye Fluo-4 and live cell imaging with confocal microscopy was established for the cells. The cells were treated with oxytocin and a Ca²⁺-influx to the cytosol was demonstrated (FIG. 2B).

The effect was dose-dependent with a maximum effect already at 0.05 IU/ml oxytocin. For the experiments DF01 as described Example 1 was used.

FIG. 2A shows that DF01 alone did not affect the Ca²⁺-concentration. However, when DF01 was given together with oxytocin, an increased and sustained Ca²⁺-level was attained compared oxytocin alone (FIGS. 2B and C). The dose response pattern, see FIG. 2D, shows that the number of Ca²⁺-peaks correlate with the concentration of DF01. The results demonstrate a mechanism for how DF01 exert an effect on uterine contraction by promoting and sustaining the effect of oxytocin. The mechanism was further investigated by preincubating uterine smooth muscle cells with 10 μM of verapamil for 30 min. Verapamil did not affect the Ca²⁺ influx, induced by either oxytocin or by the combination of oxytocin and DF01. It can therefore be concluded s that L-channels not are involved.

It was further investigated if the main transport mechanism of inositol-3 phosphate (IP3) stimulated Ca²⁺ transport of the endoplasmatic reticulum. To study this pathway, 2-Aminoethoxydiphenyl borate (2-APB) was tested on Ca²⁺ after 30 min of incubation with a concentration of 100 μM. This inhibitor decreased strongly both the oxytocin and the oxytocin/DF01 stimulated Ca²⁺-transport.

To further characterize the interaction between oxytocin and DF01 the effect of the oxytocin receptor antagonist Atosiban was used and the cells subjected to the DF01 enhanced oxytocin effect on Ca²⁺ transport. Atosiban in a concentration of 10⁻⁶ M clearly inhibited the effect of both oxytocin and the combination oxytocin/DF01

The results indicate that DF01 does not by itself affect Ca²⁺-transport. However in combination with oxytocin a clear dose response enhanced stimulation of Ca²⁺ transport is noted. DF01 stabilizes the effect of oxytocin resulting in longer periods of stimulation. The effect of does not involve L-channels but rather involves IP3 stimulated Ca²⁺ influx in oxytocin signaling. The effect of the oxytocin antagonist suggests that the effect on DF01 operates on the oxytocin receptor level.

It is concluded that DF01 and chemically modified heparins according to the invention are useful agents to administer for improving myometrial contractions and to treat complications associated with inadequate or absent myometrial contractions. In summary, DF01 and similar chemically modified heparin and heparin sulfates are regarded to be effective directly intervening treatments required to re-establish effective labor.

Although particular embodiments have been disclosed herein in detail, this has been done by way of example for purposes of illustration only, and is not intended to be limiting with respect to the scope of the appended claims that follow. In particular, it is contemplated by the inventor that various substitutions, alterations, and modifications may be made to the invention without departing from the spirit and scope of the invention as defined by the claims.

1. A kit comprising a chemically modified heparin or heparan sulfate in combination with an agent capable of promoting myometrial contractions of the uterus,
the chemically modified heparin or heparan sulfate having an antifactor IIa activity of less than 10 IU/mg, an antifactor Xa activity of less than 10 IU/mg, and an average molecular weight (Mw) from about 4.6 to about 6.9 kDa, and comprising
(i) polysaccharide chains essentially free of chemically intact saccharide sequences mediating the anticoagulant effect; and
(ii) polysaccharide chains corresponding to molecular weights between 1.2 and 12 kDa with a predominantly occurring disaccharide according to (Formula I), 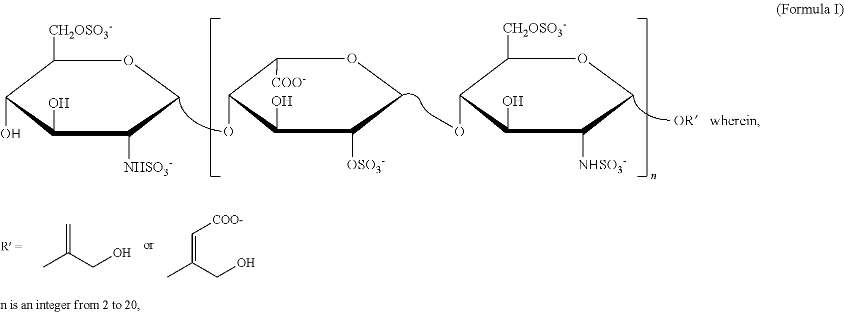
wherein the chemically modified heparin or heparan sulfate comprises non-reducing end unsaturated glucosamines presented as signals in the interval of 5.0 to 6.5 ppm in an ¹H-NMR spectrum with an intensity (% ratio) of less than 4% in relation to the signal at 5.42 ppm from native heparin.

2-7. (canceled)

8. The kit according to claim 1, wherein the agent capable of promoting myometrial contractions of the uterus is oxytocin.

9. (canceled)

10. The kit according to claim 1, wherein the predominantly occurring polysaccharide chains have between 6 and 12 disaccharide units with molecular weights from 3.6 to 7.2 kDa.

11. The kit according to claim 1, wherein at least 70% of the polysaccharide chains have a molecular weight above at least 3 kDa.

12. The kit according to claim 1, wherein the chemically modified heparin or heparan sulfate has a distribution of polysaccharides and their corresponding molecular mass expressed as cumulative % of weight according to the table:

| Molecular mass, kDa | Cumulative weight, % |
|---|---|
| >10 | 4-15 |
| >8 | 10-25 |
| >6 | 22-45 |
| >3 | >70 |

13-14. (canceled)

15. The kit according to claim 1, wherein the glucosamine signals are present at 5.95 ppm and 6.15 ppm.

16. The kit according to claim 1, wherein glucosamines comprise less than 1% of the total glucosamine content.

17-18. (canceled)

19. The kit according to claim 16, wherein the chemically modified heparin or heparan sulfate is essentially free of intact non-sulfated iduronic and/or glucuronic acids.

20. The method according to claim 23, wherein the at least one chemically modified heparin or heparan sulfate is present in a topical pharmaceutical preparation and said administering is carried out parenterally.

21. The method according to claim 23, wherein the at least one chemically modified heparin or heparan sulfate is present in a parenteral pharmaceutical preparation and said administering is carried out parenterally.

22. The method according to claim 21, wherein the chemically modified heparin or heparan sulfate is administered intravenously every 1-4 hours and the agent capable of promoting myometrial contractions is oxytoxin is administered adjunctively for up to 36 hours.

23. A method for treatment of labor arrest comprising:
administering to a pregnant woman a chemically modified heparin or heparan sulfate with an antifactor IIa activity of less than 10 IU/mg, an antifactor Xa activity of less than 10 IU/mg, and an average molecular weight (Mw) from about 4.6 to about 6.9 kDa, wherein the chemically modified heparin or heparan sulfate comprises
(i) polysaccharide chains essentially free of chemically intact saccharide sequences mediating the anticoagulant effect; and
(ii) polysaccharide chains corresponding to molecular weights between 1.2 and 12 kDa with a predominantly occurring disaccharide according to (Formula I), 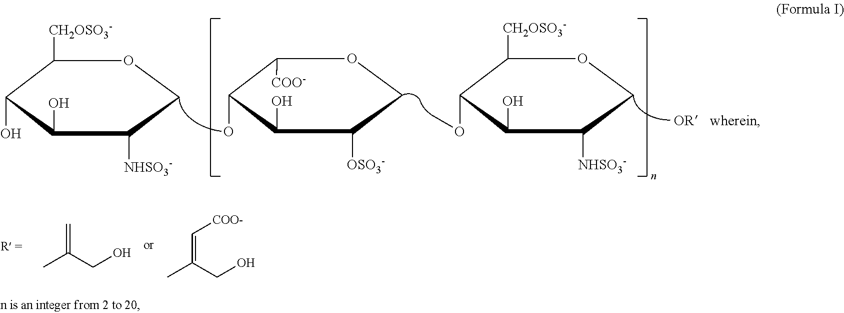
wherein the chemically modified heparin or heparan sulfate comprises non-reducing end unsaturated glucosamines presented as signals in the interval of 5.0 to 6.5 ppm in an ¹H-NMR spectrum with an intensity (% ratio) of less than 4% in relation to the signal at 5.42 ppm from native heparin; and
administering to the pregnant woman an agent capable of promoting myometrial contractions of the uterus.

24. The method according to claim 23, wherein the labor arrest is primary labor arrest.

25. The method according to claim 23, wherein the labor arrest is secondary labor arrest.

26. The method according to claim 25, wherein the secondary labor arrest is a complete cessation of progress.

27. The method according to claim 25, wherein the secondary labor arrest is due to cephalopelvic disproportion.

28. The method according to claim 23, wherein the labor arrest is in a woman who has been induced into labor.

29. The method according to claim 23, wherein the labor arrest is in a woman who is nulliparous.

30. The method according to claim 23, wherein the agent capable of promoting myometrial contractions of the uterus is oxytocin.

31. (canceled)

32. The method according to claim 23, wherein said administering the chemically modified heparin or heparan sulfate is subsequent to said administering the agent capable of promoting myometrial contractions of the uterus.
